(19) United States
(12) Patent Application Publication
(10) Pub. No.: US 2022/0175322 A1
(43) Pub. Date: Jun. 9, 2022

(54) System and Method for Adaptive Interference Suppression

(71) Applicant: Tricog Health Pte Ltd, Singapore (SG)

(72) Inventors: Udayan Dasgupta, Bengaluru (IN); Achuth Pv, Calicut (IN)

(21) Appl. No.: 17/441,455

(22) PCT Filed: Mar. 20, 2020
(86) PCT No.: PCT/SG2020/050149

§ 371 Date: Sep. 21, 2021

(30) Foreign Application Priority Data
Mar. 22, 2019 (IN) 201941011223

(51) Int. Cl. A61B 5/00; A61B 5/318; A61B 5/28

(52) U.S. Cl. CPC A61B 5/7203; A61B 5/28; A61B 5/318

(57) ABSTRACT

The disclosed invention provides a system and method for adaptive interference suppression for a signal in general and an ECG signal in particular. The method includes acquisition of a signal through an acquisition process. The signal is further passed through an FFT module configured to perform time windowing and FFT operation on the signal. The method further includes setting one or more fixed or adaptive thresholds for detecting tonal interferers in the signal. The method further includes choosing an appropriate filter for filtering the detected tonal interferers in the signal. Further, the detected tonal interferers are removed using the chosen appropriate filter. Finally, the method includes removing padding from the signal to eliminate residual edge effects.

### CROSS-REFERENCE TO RELATED APPLICATIONS

This application is the United States national phase of International Application No. PCT/SG2020/050149 filed Mar. 20, 2020, and claims priority to Indian Patent Application No. 201941011223 filed Mar. 22, 2019, the disclosures of which are hereby incorporated by reference in their entirety.

### BACKGROUND OF THE INVENTION

### Field of the Invention

The field of invention generally relates to a method for removing tonal interferers from a signal; and more specifically, it relates to a system and method for adaptive interference suppression and removal in ECG signals.

Description of Related Art

Electrocardiography (ECG or EKG) is the process of recording the electrical activity of a heart over a period of time by using electrodes placed over the skin. ECG recordings are corrupted by various types of noise. The most common forms of noise that are encountered during ECG include low frequency noise signals due to a patient's breathing and high frequency interference or noise signals due to activity of the patient's skeletal muscles. For most ECG analyses, frequency band of interest lies between 0.5 Hz to 150 Hz. Also, the most common forms of noise encountered during recording of ECG are either lower or higher in frequency in comparison with the band of interest. Typically, these most common forms of noise are either removed by low pass filtering or high pass filtering or by a combination of both.

Further ECGs also suffer from tonal interferences. Tonal interferers are commonly caused by power line interferences of 50-60 Hz and their harmonics. Tonal interferers lie within frequency range of 50 Hz to 60 Hz (band of interest), which as a consequence makes them difficult to filter. Tonal interferences include electromagnetic interferences originating from power lines in the vicinity of the ECG device. Tonal interferences make ECGs unreadable and hide subtle features of interest. Furthermore, tonal interferences lead to improper processing of ECG signals by ECG processing algorithms and can therefore results in a misreading of the ECG signal and a subsequent misdiagnosis by a physician. Hence, it is imperative that such tonal interferers be suppressed (typically via notch filtering) or filtered before further processing. Current systems apply well-designed notch filters for filtering of tonal interferers. However, residual edge effects can often remain in the signal even after such filtering. Since the residual edge effects contained within the processed ECG indicate the presence of interferers at the edge of the signal, current systems show a poor ability in removing edge effects.

Other typical approaches for suppressing or filtering said tonal interferers include using fixed notch filters and adaptive filters. Fixed notch filters are employed when the tonal interferer's characteristics are known, while adaptive filters are employed when the tonal interferer's characteristics are unknown. Infinite Impulse Response (IIR) notch filters are also popular in practice but their usage entails a risk of introducing phase distortion or ripples in the spectrum surrounding the notch frequency, and may have stability issues. A stable alternative to IIR notch filters is a Finite Impulse Response (FIR) filter, which can be configured to avoid phase distortion. However, the usage of FIR filters typically results in worse complexity-attenuation performance tradeoffs.

Traditional approaches for removing edge effects include padding (or extension) of the signal. In any padding method, a signal is pre-padded and post-padded resulting in a composite signal. Further, the composite signal is processed to avoid or remove edge effects, after which the padded portions are removed to get the filtered signal. Although the mentioned padding method reduces some edge effects, some residual edge effects remain and the issues arising out of presence of tonal interferers in a signal are not completely resolved.

Thus, in light of the above discussion, it is implied that there is a need for a scheme of signal processing which is reliable and does not suffer from the problems discussed above.

### SUMMARY OF INVENTION

The principle object of this invention is to provide a system and method for adaptive interference suppression in ECGs.

Another object of the invention is to provide a system and for removing tonal interferers (single frequency noise) in a signal.

Another object of the invention is to provide a post-processing scheme for signal processing that is configured to determine which subset of ECGs need notch filtering.

Yet another object of the invention is to provide a signal acquisition scheme that does not suffer from edge effects.

Yet another object of the invention is to minimize negative impact (phase distortion or pass-band ripples) of notch filters.

The present invention provides a system and method for adaptive interference suppression for a signal in general and an ECG signal in particular. The method comprises acquiring a signal through an acquisition process. Further, the method comprises passing the signal through an FFT module, wherein an FFT is performed on the signal. The method further comprises setting one or more fixed or adaptive thresholds for detecting tonal interferers in the signal. Subsequently, the method comprises choosing an appropriate filter for filtering the detected tonal interferers in the signal. Further, the detected tonal interferers are removed using the chosen appropriate filter. Finally, the method comprises removing padding (if any) from the signal to eliminate residual edge effects in the signal.

### BRIEF DESCRIPTION OF THE DRAWINGS

This invention is illustrated in the accompanying drawings, throughout which, like reference letters indicate corresponding parts in the various figures.

The embodiments herein will be better understood from the following description with reference to the drawings, in which:

FIG. 1 depicts/illustrates an original episodic ECG of 10 s duration, wherein the abscissa (horizontal axis) depicts time represented in milliseconds (ms) and the ordinate (vertical axis) depicts amplitude represented in millivolts (mV).

FIG. 2A depicts/illustrates the original episodic ECG of 10 s duration corrupted with tonal interferers resulting in a noisy signal.

FIG. 2B depicts/illustrates the output signal of original episodic ECG of 10 s duration wherein the bulk of the tonal interference is removed by filters.

FIG. 3 depicts/illustrates original ECG signal corrupted with OHz noise.

FIG. 4 depicts/illustrates FFT (Fast Fourier Transform) spectrum of the original ECG signal, wherein threshold interference tones are found at 50 Hz and 150 Hz.

FIG. 5 depicts/illustrates the original ECG signal filtered/cleaned by using the method disclosed by the present invention.

FIG. 6 depicts/illustrates a method for adaptive interference suppression in ECGs.

FIG. 7 depicts/illustrates m detail, a method for adaptive suppression in ECGs.

FIG. 8 depicts/illustrates a system for adaptive interference suppression in ECGs.

FIG. 9 depicts/illustrates in detail the components of the system for adaptive interference suppression in ECGs.

### DESCRIPTION OF THE INVENTION

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

ECG signals are episodic in nature and are collected under a variety of conditions, with varied interferences that can be present in the ECG signals. Different filters and filtering techniques are required for different interfering frequencies. The method disclosed herein describes an approach wherein the ECG signal is not filtered unless an interference is detected. Further, only detected interfering frequencies are removed through filtering. The disclosed method is useful to minimize negative impacts of notch filters, such as phase distortion or pass band ripples in the filtered signal. Additionally, the method also discloses an acquisition process configured to overcome edge effects completely. The current disclosure also discloses a system configured to generate distortion free outputs for signals within a band of interest comprising strong tonal interferers.

The method comprises acquiring a signal through an acquisition process. The acquisition process is configured to acquire at least one signal in the form of L+2p′, wherein the component ‘L’ is number of samples required in the signal and component ‘p’ is number of padding samples required on either end of the signal. The acquired signal is preferably an ECG signal. The method further comprises passing the signal through a (Fast Fourier Transform) FFT module for performing Fast Fourier Transform on the signal. The FFT module is further configured to time-window the signal using a time-windowing function prior to performing FFT on the signal. The time-windowing function is preferably Blackman Harris. The time windowing operation is typically used to minimize the side-lobes resulting from FFT performed on the signal.

The present invention provides a system and method for adaptive interference suppression for a signal in general and an ECG signal in particular.

FIG. 1 depicts/illustrates an original episodic ECG of 10 second duration, wherein the abscissa (horizontal axis) depicts time represented in milliseconds (ms) and the ordinate (vertical axis) depicts amplitude represented in millivolts (mV). The signal is also called as a ‘clean’ signal since the signal is not corrupted by any noise or tonal interferers.

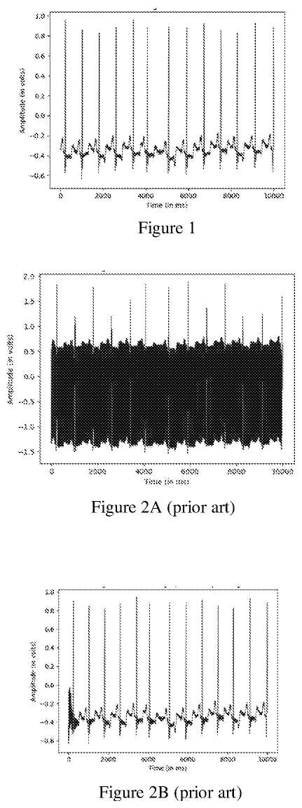

The effect of tonal interferers on an ECG signal is shown in FIG. 2A and FIG. 2B. The interferers comprise one or more of single tone interference, arising from sources such as power line, electromagnetic field and the like.

FIG. 2A depicts/illustrates an original episodic ECG of 10 second duration corrupted with tonal interferers resulting in a noisy signal.

FIG. 2B depicts/illustrates the output signal of original episodic ECG of a 10 second duration, of a prior art. In the output signal, some of the tonal interferences were removed by prior art filters. However, on close examination of the output signal in FIG. 2B, it is observed that the resulting output signal suffers from edge effects, which can be seen at the edge of the signal at Oms and prior to 10 ms.

In an embodiment, the original ECG signal depicted in FIG. 1 can be acquired through an acquisition process as described the disclosed method. The acquisition process is configured to acquire at least one signal using a formula ‘L+2p’ samples, wherein the component ‘L’ depicts the number of samples required in the signal and component ‘p’ depicts the number of padding samples required on each end of the signal. The acquired signal is preferably an ECG signal.

ECG signals are typically captured for a duration of 10 seconds in common practice. However, in the method disclosed by the current invention, the ECG signal is captured for a slightly longer duration i.e., for more than 10 seconds. In the present case, the signal is preferably recorded for duration of 12 seconds. The ECG signal that is captured for a slightly longer duration, i.e., 12 seconds instead of 10 seconds, is captured such that there is an additional recording of one second on either side of the ECG signal (i.e., ECG signal=1 second+10 second+1 second, wherein the signal in the 10 second duration is termed as a band of interest). The additional recording of 1 second on either side of the signal has an effect equivalent to pre-padding and post-padding the signal. The additional recording of 1 second on either side of the signal is advantageous because it helps in the removal of edge effects when the signal is processed. In a preferred embodiment of the invention, the ECG signal is processed by using a forward-backward algorithm, if an IIR filter is used. The forward-backward algorithm is configured to minimize phase distortion of the signal wherein IIR filters are used for filtering the signal. Further, usage of forward-backward algorithm is not required if a linear phase FIR filter is used for processing of ECG signal in the preferred embodiment.

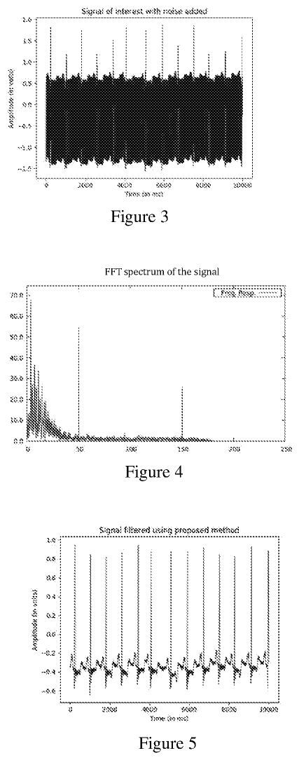

FIG. 3 depicts/illustrates an original ECG signal corrupted with 50 Hz noise, which was subsequently processed to depict the working of the disclosed method. It can be observed from the figure that the signal requires filtering to make the signal readable.

FIG. 4 depicts/illustrates an FFT spectrum of the original ECG signal depicted in FIG. 3. The signal that is acquired through the acquisition process is fed into an FFT module, wherein the signal is windowed using a time windowing function. The time windowing function is preferably Blackman Harris. Subsequent to time windowing the signal, FFT is performed on the signal resulting in an FFT spectrum as illustrated. Using a time window on the signal helps in minimizing side lobes resulting from the FFT performed on the signal. The signal (FFT spectrum) illustrated in FIG. 4 comprises tonal interferences at 50 Hz and 150 Hz. The presence of such tonal interferers emphasizes the need for an accurate filtering system and a method that can filter such tonal interferences. The tonal interferences found at 50 Hz and 150 Hz make the signal (ECG signal in the present case) unreadable, and may lead to various misdiagnosis, if they are used by physicians without necessary filtering.

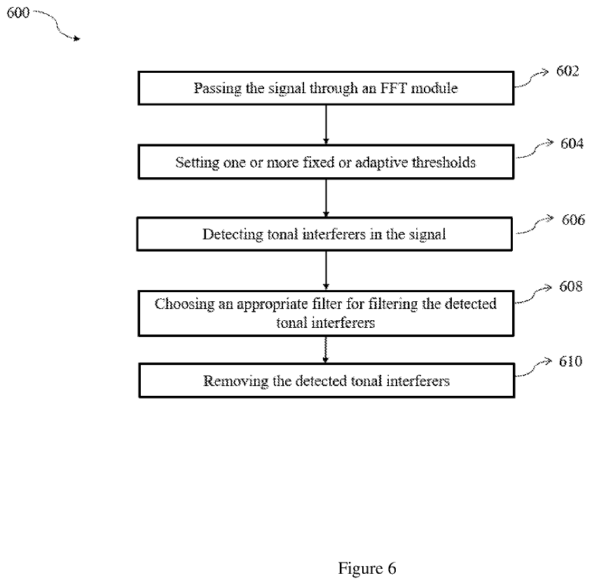

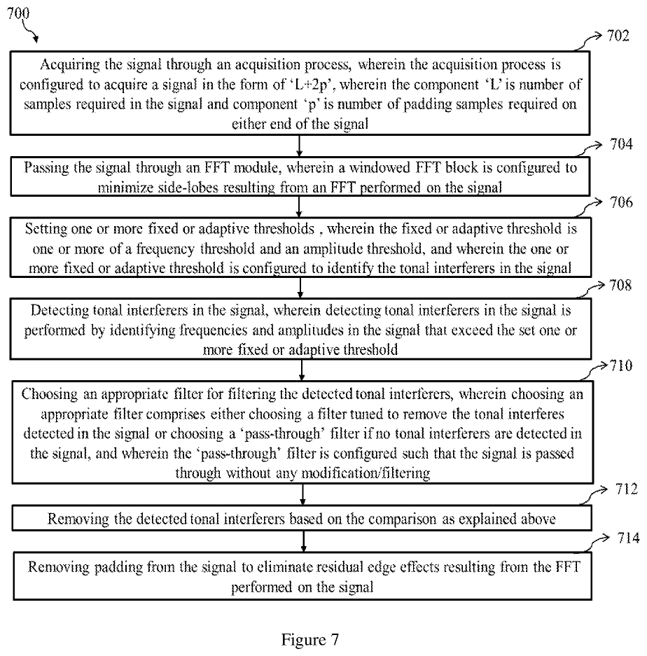

The tonal interferences at 50 Hz and 150 Hz are detected by setting one or more fixed or adaptive thresholds, as explained further in FIG. 6 and FIG. 7. Subsequently, an appropriate filter is chosen for filtering the detected tonal interferers in the signal. Finally, padding (if any) is removed from the signal to eliminate residual edge effects in the signal. Residual edge effects that may have been left in the signal as a consequence f FFT performed on the signal, are removed at this step.

FIG. 5 depicts/illustrates a resulting signal which has been filtered or cleaned using the method disclosed by the present invention. It can be observed in the figure that the filtered/cleaned signal does not contain any noise and does not suffer from any edge effects or residual edge effects.

FIG. 6 depicts/illustrates a method 600 for adaptive interference suppression in ECGs. The method 600 further comprises passing the signal through a windowed FFT module, as depicted at step 602. Subsequently, the method 600 comprises setting one or more fixed or adaptive thresholds, as depicted at step 604. Further, the method 600 comprises detecting tonal interferers in the signal, as depicted at step 606. The method 600 further comprises choosing an appropriate filter for filtering the detected tonal interferers, as depicted at step 608. The method 600 comprises removing the detected tonal interferers, as depicted at step 610.

FIG. 7 depicts/illustrates in detail a method 700 for adaptive suppression in ECGs devices. At first, the method 700 comprises acquiring a signal through an acquisition process, wherein the acquisition process is configured to acquire a signal with ‘L+2p’ number of samples, as depicted at step 702. Component ‘L’ represents a number of samples required in the signal and component ‘p’, “p” represents the number of padding samples required on each end of the signal. The method 700 further comprises passing the signal through an FFT module, wherein a windowed FFT block is used to minimize side-lobes resulting from an FFT performed on the signal, as depicted at step 704. Subsequent to step 704, the method 700 comprises setting one or more fixed or adaptive thresholds. The fixed or adaptive threshold may comprise one or more of a frequency threshold and an amplitude threshold, as depicted at step 706. Further, the fixed or adaptive threshold is used to identify the tonal interferers in the signal. The method 700 further comprises detecting tonal interferers in the signal, which is performed by identifying frequencies and amplitudes in the signal that exceed the set one or more fixed or adaptive threshold, as depicted at step 708. Furthermore, the step 708 may also comprise a search procedure, wherein the search procedure is configured to determine locations where such interferences exist. The search procedure also comprises detection of frequencies. The detection of frequencies is performed by determining whether the FFT amplitude of the detected frequencies is above a set fixed or adaptive threshold. The said search procedure is advantageous m scenarios wherein the frequency of interferers is unknown.

The method 700 further comprises choosing an appropriate filter for filtering the detected tonal interferers, as depicted at step 710. Choosing an appropriate filter comprises either choosing a filter tuned to remove the tonal interferes detected in the signal or choosing a ‘pass-through’ filter in case no tonal interferers are detected in the signal. The ‘pass-through’ filter is configured such that the signal is passed through without any modification/filtering. The method 700 further comprises removing the detected tonal interferers based on the comparison, as depicted at step 712. Finally, the method comprises removing padding from the signal to eliminate residual edge effects resulting from the FFT performed on the signal, as depicted at step 714.

In an alternate embodiment, m case the method 700 is to be applied to a continuous signal, only steps 704 to steps 712 may be carried out. In particular, steps 702 of acquiring the signal through an acquisition process and step 714 of removing the padding may not be implemented.

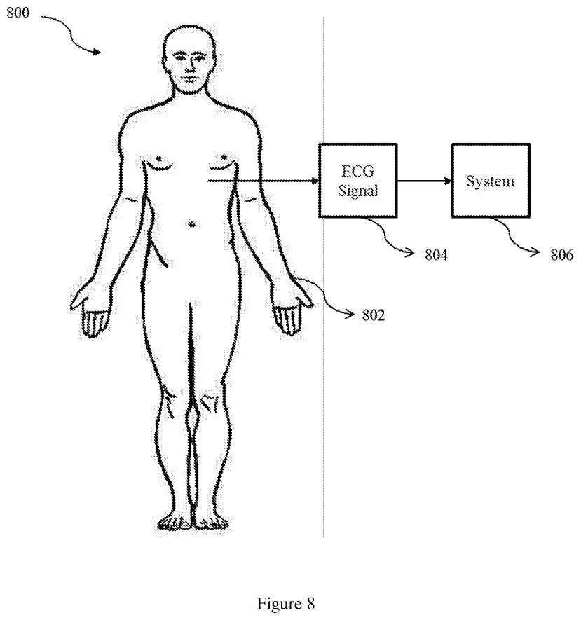

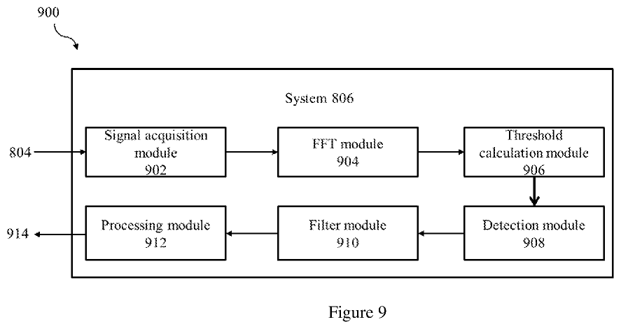

FIG. 8 depicts/illustrates an arrangement/setup 800 for adaptive interference suppression in ECG. An ECG signal 804 is acquired from a heartbeat of a user 802. The ECG signal 804 is further fed into the system 806, wherein the signal is further processed for removal or suppression of tonal interferences.

FIG. 9 depicts/illustrates in detail the components of the system 806 configured for adaptive interference suppression 900 in ECGs. The system 806 comprises a signal acquisition module 902. The signal acquisition module 902 is configured to acquire the ECG signal 804 through the acquisition process, wherein the acquisition process 1s configured to acquire the ECG signal 804 in the form of ‘L+2p’ samples. The component ‘L’ represents a number of samples required in the ECG signal 804 and component ‘p’ represents a number of padding samples required on each end of the ECG signal 804. The system 806 further comprises an FFT module 904. The FFT module 904 is configured to perform a time window function on the ECG signal 904 and is further configured to perform FFT of the ECG signal 904 to obtain FFT spectrum. In a preferred embodiment, the signal acquisition module (902) may be configured to acquire the signal in form of ‘L+2p’ samples, in case IIR filters are used for filtering the ECG signal 804. In another preferred embodiment, the signal acquisition module (902) may be configured to acquire the signal in form of ‘L+p’ samples in case FIR filters are used for filtering the ECG signal 804.

The system 806 further comprises a threshold calculation module 906. The threshold calculation module 906 comprises one or more of fixed or adaptive thresholds. The one or more fixed or adaptive threshold comprises one or more of a frequency threshold and an amplitude threshold. The fixed or adaptive threshold is used further in identifying tonal interferers in the ECG signal 804. The system 806 further comprises a detection module 908 configured to detect tonal interferers in the ECG signal 804. The detection of tonal interferers in the ECG signal 804 is performed by identifying frequencies and amplitudes in the ECG signal 804 that exceed the set fixed or adaptive threshold.

The system 806 further comprises a filter module 910. The filter module 910 is configured for choosing an appropriate filter for filtering detected tonal interferers. Choosing an appropriate filter comprises either choosing a filter tuned to remove the tonal interferes detected in the ECG signal 804 or choosing a ‘pass-through’ filter if no tonal interferers are detected in the ECG signal 804. The ‘pass-through’ filter is configured such that the ECG signal 804 is passed through without any modification/filtering. The system 806 further comprises a processing module 912. The processing module 912 is configured to remove the detected tonal interferers in the ECG signal 804. The processing module 912 is further configured to remove padding (‘2p’ component of the ECG signal 804 or any other extra padding if present) from the ECG signal 804. The processing module 912 is further configured to eliminate residual edge effects resulting from FFT performed on the ECG signal 804. The resulting ECG signal 914 can be further processed for storage, recording, display or diagnosis.

The working of the invention is explained as follows, according to a preferred embodiment of the invention. An ECG signal 804 is captured through the acquisition process described at step 702 of FIG. 7. The ECG signal 804 is captured by usage of a forward-backward algorithm. The forward-backward algorithm is configured to minimize phase distortion of the ECG signal 804 in case IIR filters are used for filtering the ECG signal 804. Subsequent to acquisition of the ECG signal 804, the ECG signal 804 is fed into the FFT module 904. The FFT module 904 time-windows the ECG signal 804 and subsequently performs FFT (implemented using an FFT) of the ECG signal 804. Time windowing operation is typically employed to minimize side-lobes resulting from an FFT operation.

Further, the ECG signal 804 is fed into the threshold calculation module 906. The threshold calculation module 906 comprises one or more fixed or adaptive thresholds for identifying or detecting tonal interferers. An adaptive threshold used as a function of a power spectrum of the ECG signal 804 is preferred. Assuming that all tonal interferers are above a particular frequency ‘F’ (F is typically around 50-150 Hz) a preferred method to set threshold ‘T’ is to configure T at a scaled down value of peak spectral component. The peak spectral component is measured within the region of primary signal content of the ECG signal 804 (typically <F).

Further, the tonal interferers are detected by the detection module 908. The detection of tonal interferers is performed by identifying frequencies and amplitudes in the ECG signal 804. Identification and detection are performed by checking for frequencies and amplitudes within the corresponding FFT spectrum, wherein the frequencies and amplitudes exceed set threshold ‘T’. The typical frequency content of an ECG is less than 45-50 Hz, 0hence search and identification of tonal interferers is carried out only at frequencies above said frequency range of 45-50 Hz.

Subsequent to the identification and detection of the tonal interferers, the ECG signal 804 is fed into the filter module 910. An appropriate filter comprised within the filter module 910 is chosen for filtering or suppressing the tonal interference. If no tonal interferers are found in the ECG signal 804, then the ECG signal 804 is considered to be “clean” and is let through a “pass-through” filter (configured not to modify the input signal in any manner). Alternately, if tonal interferers are detected, an appropriate filter comprised within the filter module 910 is chosen. Additionally, if no appropriate filter is found within the filter module 910, then the ECG signal 804 is let through a “pass-through” filter. The appropriate filter is chosen such that the filter is configured/tuned to remove the detected tonal interferes. Thus, only an appropriate amount of filtering that is required for a particular interference scenario is used on a case to case basis. Further, the ECG signal 804 is fed into the processing module 912. The processing module 912 is configured to strip off extra padding samples ‘p’ on either end of the ECG signal 804 to get a distortion free filtered output.

In another preferred embodiment, the one or more fixed or adaptive thresholds may be set either at typical interferer frequencies that are encountered in common practice (say at 50, 75, 100, 125, 150, 175, 200, Hz) or at a frequency that is indicated prominently m an FFT spectrum corresponding to the acquired ECG signal.

The filter module 910 is further configured to perform an adaptive configuration of filters for removing tonal interferences. The adaptive configuration of the filter involves configuring a filter in a manner such that the selected filter is appropriate for removing the detected tonal interference. For example, in a situation the filter module 910 comprises a filter that is suitable for filtering of tonal interferences of 50 Hz, wherein a tonal interference of 57 Hz is detected in the system. In such a situation, the filter module 910 may choose the 50 Hz filter and configure or modify the filter such that the filter is appropriate for removing a tonal interference of 57 Hz. The above-explained feature of adaptive configuration of filters is advantageous in situations wherein the detected tonal interference varies from the filters provided within the system.

In yet another preferred embodiment, the system 806 may be constructed in a form of a compact device, i.e., in a box unit. The compact device may also comprise a signal selection unit, wherein the signal selection unit further comprises the signal acquisition module 902 and the processing module 912. The compact device may be configured to acquire the signal 804 in the form of ‘L+2p’ or ‘L+p’ based on the type of filters used in the system.

In an alternate embodiment, the compact unit may be configured to be embedded or attached to a user unit, wherein the user unit may be a portable medical device, an ECG device, a smart phone, a smart watch, a fitness band, a wearable device, and the like.

In an embodiment, the compact device can be configured to monitor a user's ECG data continuously. In some embodiments wherein the compact device monitors data continuously, the signal may be acquired in the form of ‘L+2p’ or ‘L+p’, wherein the component ‘p’ is considered to be equal to zero. Alternatively, the compact device may not include the signal acquisition module 902 and the processing module 912, as the continuous signal input would not require padding and removal of padding. In a further embodiment, this continuous signal input may be shared over one or more communication networks to the system 806 for removing tonal interferers.

The advantages of the current invention include ‘selective filtering’. Selective filtering suppresses typical issues like phase or amplitude distortion in ECG signals. Further, if an ECG signal is found to be ‘clean’, then the ECG signal is progressed through a ‘pass-through’ filter, which does not modify the ECG signal in any manner. It is also pertinent to mention here that continuous filtering of ECG signals using IIR filters is not performed in the current invention, because such an approach would introduce phase distortion into ECG signals.

In an exemplary embodiment, the current invention was used to process a large variety of ECG signals collected from different ECG measurement apparatus and environments. All the collected ECG signals were at least 10 second (5000 samples) episodic ECGs collected at a sampling rate of 500 Hz. A threshold ‘T’ was used to search for and identify tonal interferers at frequencies greater than or equal to F=50 Hz (since the spectral content of the ECG signal content is typically less than 50 Hz). The threshold ‘T’ used for flagging a presence of tonal interferers was set at T=K.P, where K is a constant (set to 0.25 for testing) and P is peak magnitude response for frequencies less than F. A vast majority of the collected ECG signals were found to be ‘clean’. Such ‘clean’ ECG signals are passed unfiltered for further processing.

Often ECG signals corrupted with tonal interferers and their harmonics were encountered. Tonal interferers were frequently found at 50 Hz and 150 Hz. ECG signals with tonal interferers were passed through pre-designed filters with thresholds set at 50 Hz and 150 Hz. As expected, the current filtering method significantly removed/suppressed the tonal interferers and provided a far better representation of the ECG signal in comparison with prior art techniques. Also on examination of a large number of samples (L=10000, p=2000), it was observed that edge effects in the ECG signal can be completely removed by usage of current invention.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described here.

1. A system for adaptive interference suppression for removing tonal interferers in a signal comprising:
at least one electrode to detect an electric signal in a living being; and
a digital signal processing unit for processing the signal to remove tonal interferers, wherein the digital signal processing unit comprises:
an FFT module configured to perform FFT on the signal;
a threshold calculation module configured with one or more fixed or adaptive thresholds for detection of tonal interferers in the signal;
a threshold calculation module configured to detect tonal interferers in the signal by comparing the signal with the set one or more fixed or adaptive threshold; and
a filter module configured to choose an appropriate filter for filtering the detected tonal interferers in the signal.

2. The system of claim 1, comprising a signal selection unit, wherein the signal selection unit further comprises:
a signal acquisition module configured for acquiring the signal from the at least one electrode; and
a processing module configured to remove padding and eliminate residual edge effects in the signal.

3. The system of claim 2, wherein the signal acquisition module acquires the signal in form of ‘L+2p’ samples, wherein the component ‘L’ represents a number of samples required in the signal, and the component ‘p’ represents a number of padding samples required on each end of the signal.

4. The system of claim 2, wherein the signal acquisition module acquires the signal in form of ‘L+p’ samples, wherein the component ‘L’ represents a number of samples required in the signal, and the component ‘p’ represents a number of padding samples required on one end of the signal.

5. The system of claim 1, wherein the filter module configures a filter for a tonal interferer of a particular frequency, in case there is no filter corresponding to the said frequency.

6. The system of claim 1, wherein the threshold calculation module is configured to set one or more of a frequency threshold and an amplitude threshold.

7. The system of claim 1, wherein the detection module is configured to detect tonal interferers in the signal, and wherein the detection of the tonal interferers is through identification of frequencies and amplitudes in the signal that exceed the set one or more fixed or adaptive threshold.

8. The system of claim 1, wherein the filter module is configured to:
filter a predetermined amount required by the signal for a particular tonal interference; and
provide a distortion free filtered output of the signal.

9. The system of claim 1, wherein the filter module is configured to pass the signal through a ‘pass-through’ filter in case the signal does not comprise any tonal interferers.

10. A method for adaptive interference suppression in a signal, wherein the method comprises:
using at least one electrode to detect an electrical signal in a living being; performing a Fast Fourier Transform on the signal by passing the signal through an FFT module;
setting one or more fixed or adaptive thresholds for detection of tonal interferers in the signal by using a threshold calculation module;
detecting tonal interferers in the signal by comparing the output of the FFT module with the one or more thresholds, by using a detection module;
choosing an appropriate filter for filtering the detected tonal interferers, by using a filter module; and
removing the detected tonal interferers by using the chosen appropriate filter.

11. The method of claim 10, wherein the method comprises:
acquiring the signal from the at least one electrode through an acquisition process by using a signal selection unit comprising a signal acquisition module; and
removing padding from the signal to eliminate residual edge effects in the signal, by using a processing module.

12. The method of claim 11, wherein the acquisition process comprises acquiring the signal through a signal acquisition module such that the signal is in the form of ‘L+2p’, wherein the component ‘L’ represents a number of samples required in the signal, and the component ‘p’ represents a number of padding samples required on each end of the signal.

13. The method of claim 10, wherein the method comprises:
setting one or more of a frequency threshold and an amplitude threshold during the setting of the one or more fixed or adaptive thresholds, by using a threshold calculation module; and
identifying the tonal interferers in the signal by using the one or more fixed or adaptive thresholds.

14. The method of claim 10, wherein the detecting comprises identifying frequencies and amplitudes in the signal that exceed the one or more fixed or adaptive thresholds, wherein the detecting is implemented by using a detection module.

15. The method of claim 10, wherein choosing an appropriate filter comprises choosing a filter tuned to remove the tonal interferers detected in the signal or choosing a ‘pass-through’ filter in case no tonal interferers are detected in the signal, and wherein the ‘pass-through’ filter is configured to pass the signal without any filtering or modification.

16. The method of claim 10, comprising:
filtering a predetermined amount required by the signal for a particular tonal interference by using the chosen appropriate filter; and
providing a distortion-free filtered output of the signal.

17. The method of claim 10 comprising passing the signal through a ‘pass-through’ filter in case the signal does not comprise any tonal interferers, by using a filter module.
